(19) United States
(12) Patent Application Publication
(10) Pub. No.: US 2013/0085550 A1
(43) Pub. Date: Apr. 4, 2013

(54) MEDICAL IMPLANT RANGE EXTENSION BRIDGE APPARATUS AND METHOD

(71) Applicants: Richard J. Polefko, Parma, OH (US); Steven Wilder, Ashland, OH (US)

(72) Inventors: Richard J. Polefko, Parma, OH (US); Steven Wilder, Ashland, OH (US)

(73) Assignee: GREATBATCH, LTD., Clarence, NY (US)

(21) Appl. No.: 13/250,283

(22) Filed: Sep. 30, 2011

(51) Int. Cl. A61N 1/08

(52) U.S. Cl. 607/59; 607/60

(57) ABSTRACT

A medical device for use in providing therapy to a patient by bridging or otherwise extending the range of an external device for wirelessly connecting to a medical device, such as an implanted medical device for providing stimulation therapy to a patient

### FIELD OF THE INVENTION

This application relates generally to a medical device for use in supporting providing therapy to a patient. More specifically, this application relates to a device for bridging or otherwise extending the range of an external device for wirelessly connecting to an implanted medical device, such as a medical device for providing stimulation therapy to a patient, for example.

### BACKGROUND OF THE INVENTION

Medical devices for providing therapy to patients are becoming more commonplace. For example, neurostimulation devices deliver therapy in the form of electrical stimulation pulses to treat symptoms and conditions, such as chronic pain, Parkinson's disease, or epilepsy, for example. Implantable neurostimulation devices, for example, deliver neurostimulation therapy via leads that include electrodes located proximate to the muscles and nerves of a patient. Treatments frequently require a number of external devices, such as one or more neurostimulator controller devices, programming devices, and a neurostimulation device charger when the device utilizes a rechargeable battery. Neurostimulator controllers and programmers are frequently used to adjust treatment parameters, select programs, and even to program treatment platforms into the implantable device. External neurostimulator device chargers are used to recharge batteries on the implanted device.

Conventional neurostimulator controllers are approximately the size of hand-held gaming system controllers, smartphones, or PDAs, and may wirelessly connect to the implanted medical device, such as by utilizing inductive or RF (ISM) wireless communications technologies.

Medical devices may utilize a wireless technology that is an industry standard, such as the Medical Implant Communication Service (MICS), a specification using a frequency band between 402 and 405 MHz in communication with medical implants, and the more recently developed Medical Device Radiocommunication Service (MedRadio), which is intended to replace MICS. MedRadio maintains most of the technical rules of the MICS service. MedRadio keeps the spectrum previously allocated for MICS (402-405 MHz), but adds additional adjacent spectrum (401-402 MHz and 405-406 MHz). MICS and MedRadio allow bi-directional radio communication with devices such as pacemakers, neurostimulation devices, or other electronic implants. The maximum transmit power is very low with an EIRP of about 25 μW, in order to reduce the risk of interfering with other users of the same band. The maximum used bandwidth at any one time is about 300 kHz, which makes it a relatively low bit-rate system when compared to Wi Fi or Bluetooth, for example. MedRadio is used for wireless implant communication because its frequency range is especially suited for wireless transmission through body tissue and is an internationally recognized frequency range for implant communication.

However, standards such as MICS and MedRadio often provided limitations that make their use less practical. For example, MICS provides for communication distances that are about two meters or less between the communicating devices. This short distance may be acceptable or even desirable in certain situations, where interference between equipment is to be minimized and where the equipment is all contained in or near the implant, such as when such devices are all contained in a sterile environment (e.g., an operating room) or otherwise in near proximity with the patient (e.g., the patient holding a communication device, or a clinician holding a communication device near the patient, such as in a physician's office). However, such distance limitations also present a disincentive on the use of standards such as MICS in real world applications when it might be desirable that an remote device (such as a computer, or a stimulator controller or programmer device) utilizing the MICS standard is to communicate with another medical device (such as an implanted MICS device like a neurostimulator) over a desired distance of more than two meters, in particular in situations where interference with other devices is not a reasonable concern or where there is insufficient room within the transmission radius to include all of the necessary equipment and their operators.

Accordingly, a means of effectively increasing the communication distance between communicating devices, such as two devices utilizing MICS or MedRadio is desirable. Also desirable would be the ability to adapt devices to using different protocols, such as adapting a Bluetooth device to communicate with a MICS or MedRadio device, for example, or for use in bridging the medical device to other networks.

### SUMMARY OF THE INVENTION

Provided are a plurality of embodiments the invention, including, but not limited to, an apparatus for providing communication between an implant in a sterile environment and an external device, comprising: a first transceiver for wireless communication with the implant using a first transmission protocol; and a second transceiver for wireless communication with the external device using a second transmission protocol.

Such an apparatus can be adapted for connecting the implant to the external device by bridging the first protocol and the second protocol, such that the first transmission protocol is a protocol restricted to a short range near the implant, and further such that the apparatus is adapted to be sterilized for placing in the sterile environment.

Also provided is an apparatus for providing communication between an implant using a short-range medical device communication protocol and a remotely located external device, comprising: a first transceiver for wireless communication with the implant using a first transmission protocol; and a second transceiver for communication with the external device using a second transmission protocol over a communication network.

Such an apparatus can be adapted for connecting the implant to the external device by bridging the first protocol and the second protocol, and such that the first transmission protocol is a protocol restricted to a short range near the implant, and further such that the apparatus is adapted to be sterilized for placing in the sterile environment.

Still further provided is an apparatus for providing communication between an implant using a short-range medical device communication protocol and a remotely located external device, comprising: a first transceiver for wireless communication with the implant using a first transmission protocol; and a second transceiver for communication with the external device using a second transmission protocol over a communication network.

Such an apparatus can be adapted for connecting the implant to the external device by bridging the first protocol and the second protocol, and such that the first transmission protocol is a protocol restricted to a short range near the implant, and further such that the apparatus is adapted to communicate a heartbeat signal between the implant and the external device.

Further provided is a system for updating a program in a medical device, comprising: a medical device for providing therapy to a patient; a programming device adapted for remotely programming a medical device; and an apparatus for providing communication between the medical device using a short-range medical device communication protocol and the programming device located remotely from the medical device.

The apparatus for providing communication further including: a first transceiver for wireless communication with the medical device using a first transmission protocol; and a second transceiver for communication with the programming device using a second transmission protocol over a communication network.

Such an apparatus can be adapted for connecting the medical device to the programming device by bridging the first protocol and the second protocol, such that the first transmission protocol is a protocol restricted to a short range near the medical device; and such that the system is adapted to provide a heartbeat signal between the medical device and the remotely located programming device; and further such that the medical device is adapted to enter a safe mode during remote programming by the remotely located programming device if the heartbeat signal is corrupted, lost, or otherwise interrupted for a defined period.

Also provided is a method for bridging communication between an implant using a medical device communication protocol in a sterile environment and an external device using a second communication protocol outside of the sterile environment, comprising the steps of:
sterilizing a bridging device comprising a first transceiver for wireless communication with the implant using the medical device communication protocol and also comprising a second transceiver for wireless communication with the external device using a second transmission protocol;
placing the sterilized bridging device in or near the sterile environment near the implant; and
using the bridging device for providing a wireless communication path between the implant and the external device that is outside of the sterile environment.

Further provided is method for bridging communication between an implanted medical device and a communication device, comprising the steps of:
placing a bridging device near a patient having a surgical procedure, wherein the patient has an implanted medical device embedded or being embedded in the patient;
the bridging device wirelessly communicating with the implanted medical device during or just after the surgical procedure; and
The bridging device also wirelessly communicating with a communication device provided outside of the sterile environment, such that the bridging device wireless communication provides a wireless communication path between the implant and the communication device that is outside of the sterile environment.

Also provided is a method of remotely programming a medical device, comprising the steps of:
establishing a communication path between the medical device and the remotely located programming device;
establishing a heartbeat between the medical device and the remotely located programming device;
remotely programming the medical device; and
monitoring the heartbeat at the medical device such that if the heartbeat is corrupted, lost, or otherwise interrupted for a defined period, the medical device enters a safe mode.

Further provided is a method of remotely programming a medical device, comprising the steps of:
establishing a communication path between the medical device and the remotely located programming device;
establishing a heartbeat between the medical device and the remotely located programming device;
remotely programming the medical device;
establishing a communication link between a patient receiving therapy from the medical device and a clinician operating the programming device such that the clinician can monitor a condition of the patient while programming the medical device;
monitoring the heartbeat at the medical device such that if the heartbeat is corrupted, lost, or otherwise interrupted for a defined period, the medical device enters a safe mode; and
providing a mechanism for the clinician to cause the medical device to enter the safe mode by manual activation by the clinician.

Also provided is a method of providing therapy to a patient, comprising the steps of:
providing an ambulatory medical device to the patient for use by the patient for providing therapy to the patient;
establishing a communication path between the medical device being used by the patient and a remotely located programming device that is not operated by the patient; and
remotely programming the medical device for updating the therapy.

Still further provided is a method of providing therapy to a patient, comprising the steps of:
providing an ambulatory medical device to the patient for use by the patient for providing therapy to the patient;
establishing a communication path between the medical device being used by the patient and a remotely located programming device that is not operated by the patient;
remotely programming the medical device for updating the therapy;
establishing a heartbeat between the medical device and the remotely located programming device; and
monitoring the heartbeat at the medical device such that if the heartbeat is corrupted, lost, or otherwise interrupted for a defined period, the medical device enters a safe mode.

Further provided is a method of providing therapy to a patient, comprising the steps of:
implanting a medical implant in a patient where at least a portion of the patient is in a sterilized environment;
providing a bridging device in or near the sterilized environment, the bridging device comprising a first transceiver for wireless communication with the implant using a first communication protocol and also comprising a second transceiver for wireless communication with an external device using a second communication protocol;
placing the external device outside of the sterile environment; and
using the external device for programming a therapy into the medical implant while the at least a portion of the patient is in the sterile environment by using the bridging device for providing a wireless communication path between the implant and the external device that is outside of the sterile environment.

Also provided are additional embodiments of the invention, some, but not all of which, are described hereinbelow in more detail.

### BRIEF DESCRIPTION OF THE DRAWINGS

The features and advantages of the examples of the present invention described herein will become apparent to those skilled in the art to which the present invention relates upon reading the following description, with reference to the accompanying drawings, in which:

FIGS. 1A and 1B are illustrations of a patient's spine with an exemplary electrical stimulator treatment system disposed to treat a particular region of the spine in accordance with one aspect of the present disclosure;

FIG. 2 is a simplified block diagram showing one example implementation of an example extender with potential external devices;

FIG. 3 is a more detailed schematic block diagram of an example extender device communicating with an implanted medical device;

FIG. 4 is a schematic block diagram of an example embodiment of the example extender device;

FIG. 5 is a block diagram showing various external devices that may connect to the example extender device;

FIG. 6 is a schematic of a use of an example extender device in an operating room;

FIG. 7 is a block diagram of an showing an extender device application for remotely linking a medical device of a patient to a remote programmer; and

FIG. 8 is a block diagram of the process of a patient locally controlling application of therapy of a medical device whose parameters were set by a remote programmer.

### DETAILED DESCRIPTION OF THE EXAMPLE EMBODIMENTS

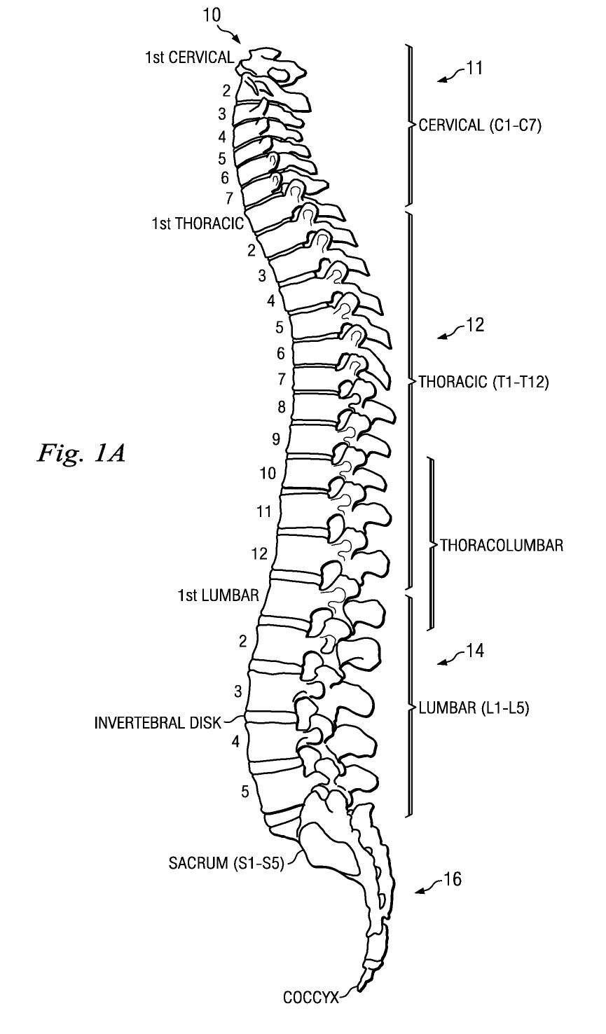

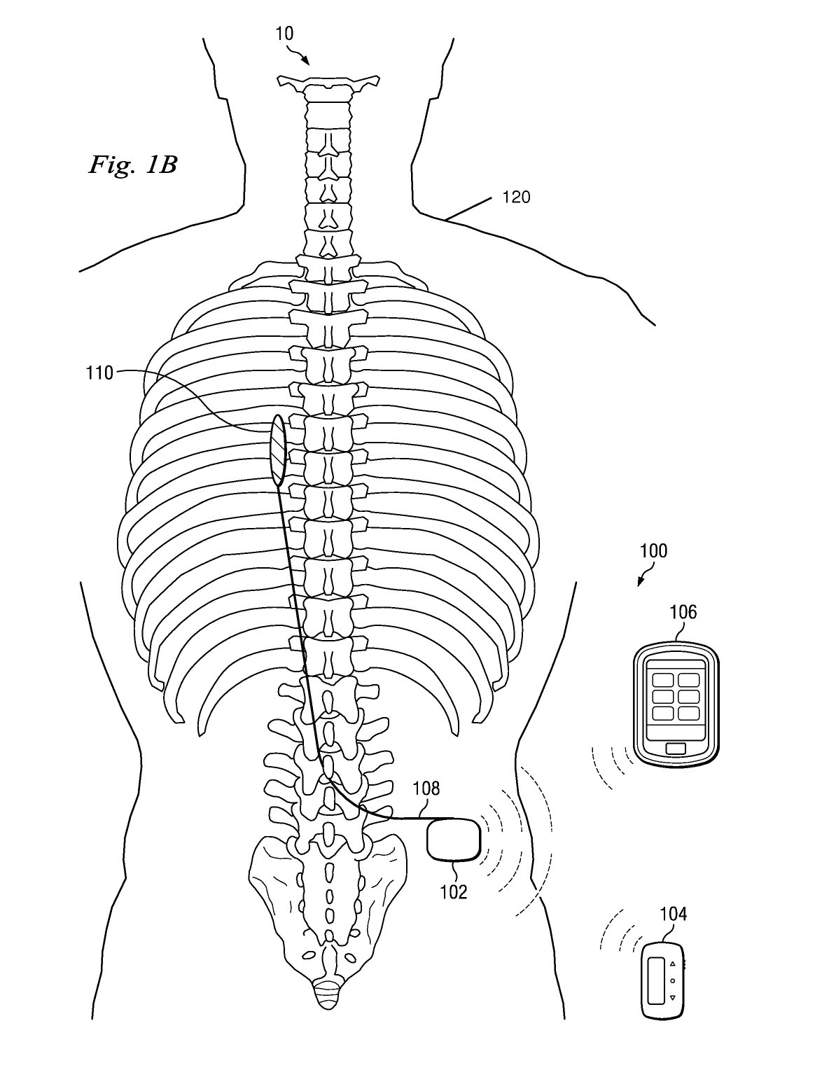

FIG. 1A is a side view of a spine 10 standing alone, and FIG. 1B is a posterior view of the spine 10 in a patient 120. FIG. 1B shows an exemplary electrical stimulator treatment system 100 disposed to treat a spinal region for treating a symptom, such as chronic pain, of the patient 120. The system includes an implantable pulse generator (IPG) 102 that delivers electrical stimulation therapy to the patient, and dual patient controllers shown and described as a pocket controller 104 and a patient controller charger (PPC) 106.

Referring again to FIGS. 1A and 1B, the spine 10 includes a cervical region 11, a thoracic region 12, a lumbar region 14, and a sacrococcygeal region 16. The cervical region 11 includes the top seven vertebrae, which may be designated with C1-C7. The thoracic region 12 includes the next twelve vertebrae below the cervical region 11, which may be designated with T1-T12. The lumbar region 14 includes the final five “true” vertebrae, which may be designated with L1-L5. The sacrococcygeal region 16 includes nine fused vertebrae that make up the sacrum and the coccyx. The fused vertebrae of the sacrum may be designated with S1-S5.

Neural tissue (not illustrated for the sake of simplicity) branches off from the spinal cord through spaces between the vertebrae. The neural tissue, along with the cord itself, can be individually and selectively stimulated in accordance with various aspects of the present disclosure. For example, referring to FIG. 1B, the IPG 102 is implanted inside the body. A conductive lead 108 is electrically coupled to the circuitry inside the IPG 102. The conductive lead 108 may be removably coupled to the IPG 102 through a connector, for example. A distal end of the conductive lead 108 is attached to one or more electrodes 110. In the example shown, the electrodes 110 are implanted adjacent to a desired nerve tissue in the thoracic region 12. The distal end of the lead 108 with its accompanying electrodes may be positioned beneath the dura mater such as by using well-established and known techniques in the art.

The electrodes 110 deliver current drawn from the IPG 102, thereby generating an electric field near the neural tissue. The electric field stimulates the neural tissue to accomplish its intended functions. For example, the neural stimulation may alleviate pain in an embodiment. In other embodiments, a stimulator as described above may be placed in different locations throughout the body and may be programmed to address a variety of problems, including for example but without limitation; prevention or reduction of epileptic seizures, bladder control, weight control, or regulation of heart beats.

It is understood that the IPG 102, the lead 108, and the electrodes 110 may be implanted completely inside the body, may be positioned completely outside the body, or may have only one or more components implanted within the body while other components remain outside the body. When implanted inside the body, the implant location may be adjusted (e.g., anywhere along the spine 10) to deliver the intended therapeutic effects of spinal cord electrical stimulation in a desired region of the spine. The IPG 102 in this example system is a fully implantable, battery-powered neurostimulation device for providing electrical stimulation to a body region of a patient. In the example shown in FIG. 1B, the IPG 102 is configured to provide neural stimulation to the spine. However, in other embodiments, IPG 102 may be a different type of pulse generator, including, for example, a pacemaker, a defibrillator, a temporary trial stimulator, or any other type of medical device. In this example, the IPG 102 is structurally configured and arranged for wireless programming and control through the skin of the patient. Accordingly, it includes a transmitter and receiver capable of communicating with external programming and control devices, such as the pocket controller 104, the PPC 106, and even a separate clinician programmer (not shown). It also includes a rechargeable power source, such as a battery configured to be wirelessly recharged through the patient's skin when the PPC 106 is externally placed in the proximity of the IPG 102.

The pocket controller 104 can provide more limited functionality relative to the functionality of the PPC 106 for controlling the IPG 102, and could thereby allows a user to control the most-used, such as daily-used, functions of the IPG 102. The PPC 106 performs all the functions of the pocket controller 104, but also includes more advanced features and functionality for controlling the IPG 102 that are used less frequently than a daily basis, such as, for example, perhaps weekly. In addition, the PPC 106 can include an integrated charger for recharging the power source in the IPG 102. The PPC 106 can be left at home, as its functions are typically not required for daily use. A separate clinician programmer (not shown) is a device typically maintained in a health care provider's possession and can be used to program the IPG 102 during office visits. For example, the clinician controller can define the available stimulation programs for the device by enabling and disabling particular stimulation programs, can define the actual stimulation programs by creating defined relationships between pulses, and perform other functions. Such a system is disclosed in U.S. patent application Ser. Nos. 13/170,775 and 13/170,558, incorporated herein by reference.

However, the system shown in FIGS. 1A and 1B are often constrained by communications limitations that are imposed by the communications protocols chosen for wireless communications between the devices. In particular, the distances at which the PPC 106 or the clinician programmer can communicate with the IPG 102 are typically limited by the chosen protocol, such as MICS or MedRadio, for example.

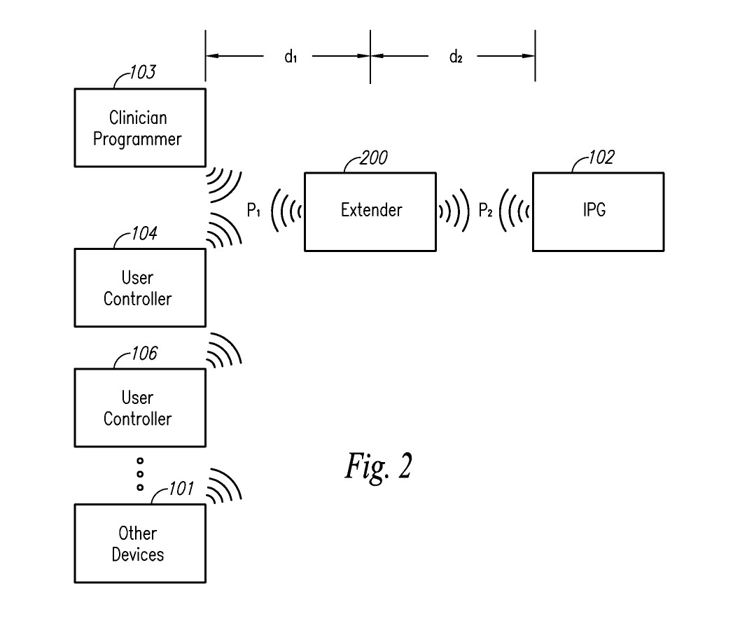

FIG. 2 shows a simplified block diagram of an example treatment system using an extender device 200 to extend the range of communication of a clinician programmer 103, a user controller 104, 106, or some other device(s) 101, for communicating with an implanted medical device, such as an IPG 102. The extender 200 extends the range of communication to a total distance of d₁+d₂ in situations where the distance d₂ may be at or near the maximum distance supported by the communication protocol used by the implantation system (e.g., d₂*two meters for a MICS system, whereas (d₁+d₂) two meters using the extender 200). The extender 200 can communicate with one or more of the remote external devices 101, 103, 106 using communication protocol P₁, and can communicate with the IPG using communication protocol P₂, where P₁ and P₂ may be the same protocol, or the extender 200 may even operate as a bridge, where P₁ and P₂ are different communication protocols, as discussed in more detail hereinbelow. Alternatively, the extender device 200 may also act as a bridge by connecting to an external device in a wired manner (e.g., USB, Ethernet, POTS, etc.), and wirelessly communicating with the IPG 102, thereby giving wired devices or networks wireless capability.

Examples of additional remote devices 101 that might communicate with the extender 200 (and thereby communicate with the IPG 102) include cellular phones, PDAs, personal computers (PCs), tablets, or even other devices more remotely connected (such as via cellular networks or via the Internet, for example).

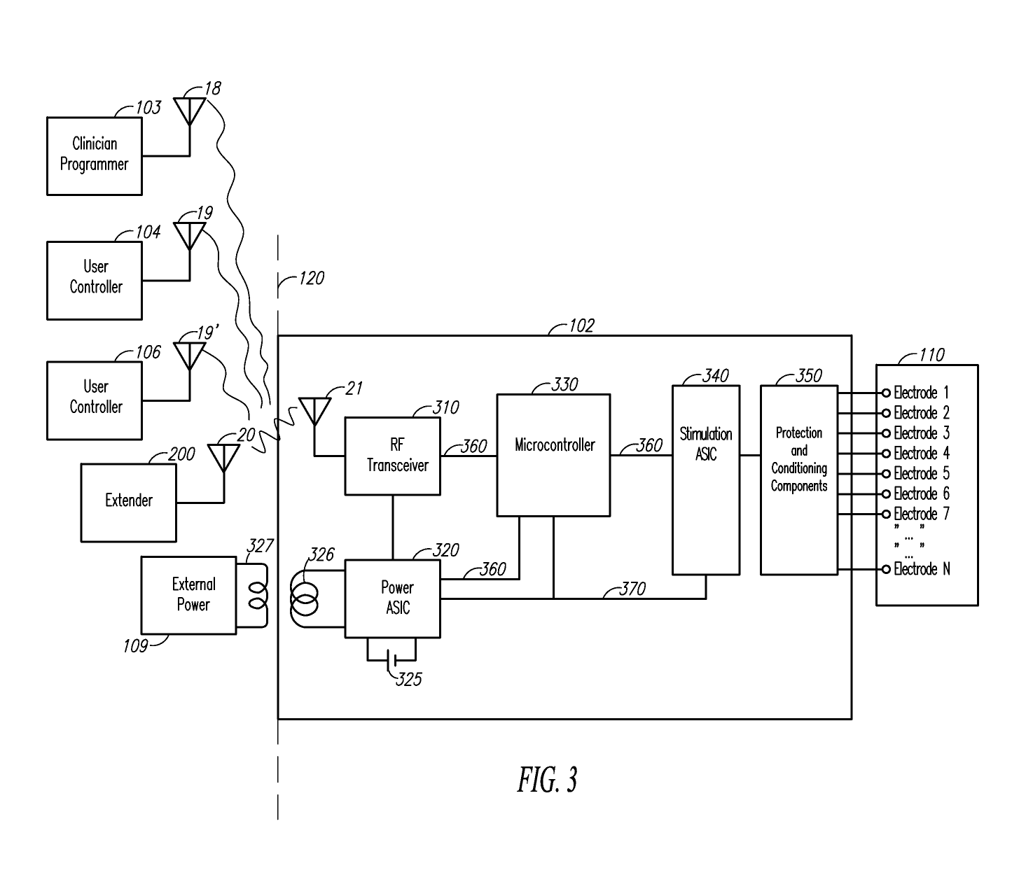

FIG. 3 shows a block diagram of a more detailed example system where most of the components for generating the stimulation waveform are implanted in a patient for providing medical therapy by utilizing an implantable PG (IPG) 102 that could utilize the disclosed features. This system is comprised of the IPG 102 that includes stimulation ASIC 340 and protection components 350. The IPG 102 is further comprised of a microcontroller 330 for controlling the functions of the IPG via the control bus 360, and a power ASIC 320 for powering the components via a power bus 370 (which also powers the RF transceiver 310). Because this implantable system avoids the need for any components or wires that exit the body of the patient 120, the IPG 102 includes an RF transceiver (transmitter/receiver) 310 with an antenna 21 for allowing the IPG to communicate with devices external to the patient's body, such as the extender 200 and the clinician programmer 103 and user controller(s) 104,106 (which may connect via the extender 200, as discussed in more detail hereinbelow) which also have antennas 20, 18 and 19, respectively, to communicate with the transceiver 310 via a wireless protocol such as MICS or MedRadio. Furthermore, the example IPG 102 also includes an embedded power supply including a power ASIC 320 for conditioning the device power, a (long life) rechargeable battery 325, and a secondary inductive coil 326 (or some other means) for wirelessly receiving power from an external source outside the body of the patient 120. A corresponding external power supply 109 would typically require a corresponding primary charging coil 327 to complete the power connection to the embedded power supply to charge the battery 325. The IPG 102 is connected to one or more electrode arrays 110 including a plurality of electrodes via a header (not shown) connected via feedthroughs (not shown) to the protection components 350. The IPG 102 is provided in a hermetically sealed case made of, or coated with, human implantable compatible materials, and such that the contacts attached to the lead body of the electrode array(s) are electrically connectable to the IPG through the header. The electrode leads and electrodes themselves, along with portions of the header that are exposed to the patient, should preferably all be made of, or coated by, materials that are compatible with implantation in the human body.

FIG. 4 shows an example embodiment of the extender 200. This example has a receiver/transmitter 220 for connecting to the implanted medical device (such as an IPG), such as for using the industry standard communication protocol (e.g., MICS or MedRadio). It also has a power supply 210 that may utilize a battery 215 (such as a rechargeable battery), or the power supply may connect to a line voltage and not require a battery. The power supply is for powering the internal components of the extender 200. An extender that is self powered (preferably from a rechargeable battery source) can be beneficial in that it requires no external connections, potentially allowing it to be subject to sterilization and useable within a sterile operating field without requiring wired connections to external devices or power lines. The device could be recharged wirelessly, such as by using an inductive charger, and the device could be encapsulated in a protective enclosure during such charging to ensure that it remain sterile.

The Extender 200 may have one or more additional receiver/transmitters 240 that may utilize a different communication protocol than the receiver/transmitter 220 (e.g., one or more of Bluetooth, Wi Fi, DECT, etc.), for allowing the extender 200 to act as a bridge to devices and/or networks using communications protocols other than that used by the implanted medical device. The example extender 200 shown in FIG. 4 will also typically have a user interface 235 for accepting user commands including at least an on/off command (e.g., a switch). Such commands may be entered by push buttons, rocker switches, or by using a menu-based system such as via an LCD screen and tracking device, or a touch screen, or some combination of these. Alternatively, the interface could be provided virtually, such as by using a web-based protocol to control the extender 200 via a remote machine, such as a computer. In such a case, the user UF would likely include some form of web server embedded therein. Other types of commands might include a safety shut-off or override button, for example. In addition, the extender could provide a mechanism (such as a special button or command) that would result in the MICS or MedRadio implant being placed into a known safe state, should a communication failure exist between the external device and the extender.

The user interface 235 may be used to support the pairing of the extender 200 to the devices to which it will connect, such as in a manner similar to the process used to pair Bluetooth devices, or cordless phones with their bases, for example.

The example extender 200 may optionally also have an external device interface 250 for connecting to external devices in a wired manner. Such an interface might include one or more of a USB interface, an Ethernet interface, a FireWire interface, and/or other types of wired interfaces, for example.

There is also a processor 230 including memory provided in the example extender for controlling the components of the extender 200. This control could include accepting and implementing user commands, selecting the correct communication protocol for communicating with selected external devices, performing self-checks on the device, storing device settings, etc. The processor may accept firmware updates from remote locations, such as over the internet via a Wi Fi connection or a wired Ethernet connection, for example.

Thus, with any of the above various optional configurations, the extender device can be utilized in a flexible manner for a number of different applications. Furthermore, a flexible device could be provided by including many or all of the options in a configurable manner, and allowing the user to choose which of the options are utilized in a given application. For example, a commercially available device with wide-ranging uses could include the option of supporting a plurality of communication protocols, such as MICS, MedRadio Wi Fi, Bluetooth, Ethernet and USB, for example. Such a device might therefore have three separate receiver/transmitters, one for each of the wireless protocols, and also a wired USB interface. Alternatively, the device might utilize a single receiver/transmitter capable of supporting all of these protocols, when and if such a component is made available, because in situations where only MICS or MedRadio supported equipment is to be utilized, a single receiver/transmitter might be sufficient. Furthermore, any of the optional components might be left off an embodiment to save on cost or size of the device, where desired.

The extender can be made disposable or reusable (sterilized) for use in a sterile operating environment, for example whereas alternative embodiments are meant to be used outside of the operating environment, such as in a doctor's office or a patient's home, and sterilization may not be necessary. Sterilization options are discussed in more detail below.

As discussed above, the extender 200 can be used to extend the effective range of communication for a MICS or MedRadio based device beyond the ˜2 meter distance inherent as a result of the standards. The end device/user controlling or exchanging data with an implanted medical device or other device can therefore be separated from the MICS or MedRadio using device at distances substantially greater than 2 meters by using the extender 200. The extended distances could be several tens, or even hundreds, of meters, using existing communication protocols (such as Bluetooth, Wi Fi, etc.). A plurality of extenders could be used to daisy-chain MICS or MedRadio protocols, for example, by placing a number of extenders at 2 meter intervals to obtain the desired communication extension.

As also discussed above, the extender 200 with the appropriate options can also be used to translate (bridge) communication with a MICS or MedRadio device to an alternative communication protocol or mechanism used by another (external) device that does not include a MICS or MedRadio transceiver. The end device/user controlling or exchanging data with an implant can therefore not be required to implement a MICS or MedRadio transceiver, and thus legacy devices, non-specialized COTS (e.g., mass market) devices, and other devices using different communication standards can be supported with MICS or MedRadio capable implants by using such an extender. Such supportable devices include: tablets, iPads, laptops, desktops, cell phones, etc., using non-medical specific communication protocols (e.g., Bluetooth, Ethernet, Wi Fi, USB, RS-232

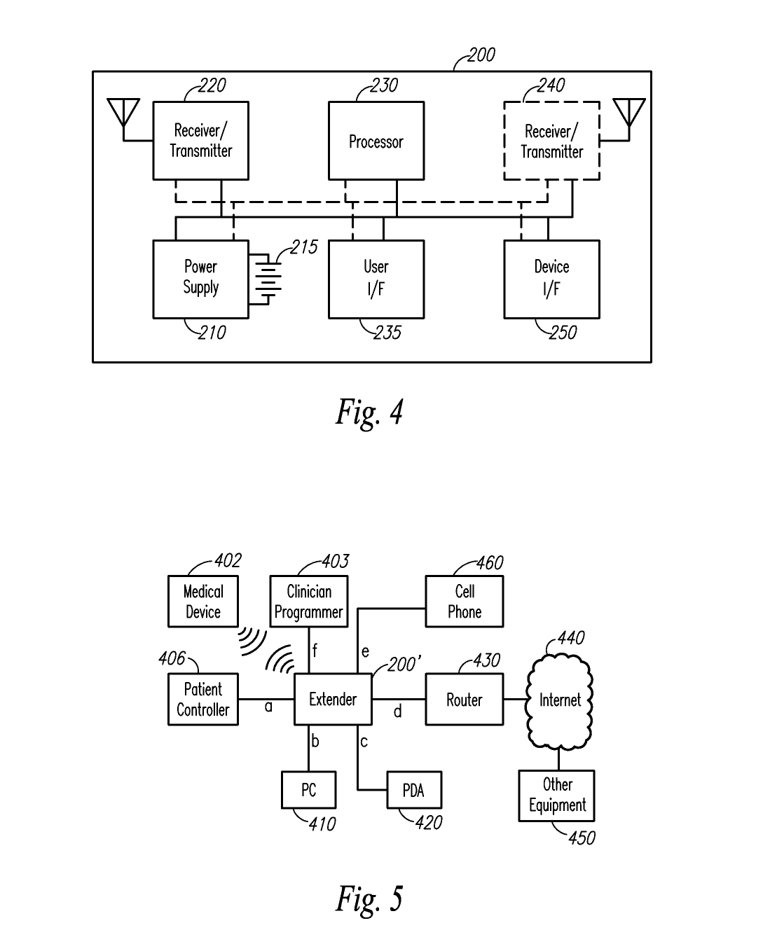

FIG. 5 shows an example general conceptual use of an example extender as described above that might be commercially available, having a number of the optional features discussed above incorporated in the device. In this example, the example extender 200′ wirelessly connects with a medical device 402 to foster communication with various external remote devices, such as a clinical programmer 403 (via communication link f), a cell phone 460 (via communication link e), a PDA 420 (via communication link c) a patient controller 406 (via communication link a), a personal computer 410 (via communication link b), and/or to a router 430 (via communication link d). Any of the communication links a-e might be wired or wireless, as desired, although links a, c, e, and f to the patient controller 406, the PDA 420, the cell phone 460, and the clinicial programmer 403 are more likely to be selected to be wireless (using such options as WiFi, or Bluetooth, or MICS, or MedRadio, or a combination thereof).

In contrast, communication links b (to the PC 410) and d (to the router 430) are likely to either be direct Ethernet connections, or WiFi connections. The connection to the router 430 allows the extender 200′ to connect to the internet 440, and thereby to other equipment 450 that is connected to the Internet. Such a connection can allow for firmware updates for the extender 200′, and/or it can allow for remote connections to the medical device 402, such as to a doctor's or clinician's office to allow for remote interaction with the medical device 402 (such as for monitoring, programming and/or updating of the medical device).

The extender can make upgrading remote equipment used with an implant much easier and more flexible, as it allows migration of legacy devices that may not yet support MICS or MedRadio standards to be used until such devices are marketed. It also will allow the communication of legacy medical devices that use the MICS or MedRadio standard with newer devices that may use a newer standard at some point in the future.

Sterile Field Applications:

Furthermore, providing wireless communication to an external device at a greater distance than the MICS or MedRadio standard reduces a number of issues that may arise when communication with the implant is required at the time the device is implanted. At the time of implantation, a sterile area (field) must be maintained around the area of the surgery. This requires adherence to aseptic procedures to ensure the sterile field and places a burden on personnel and equipment allowed within or permitted to cross the field. Any object within that area needs to be sterile. The increased communication distances provided by using an extender potentially reduces the need for sterilization of one or more of the external devices communicating with the implant, or parts of those devices, as they can remain outside the sterile field yet still connect to the implant via an extender.

Depending on the area of the sterile field, positioning of the patient, equipment, personnel, etc. requiring that the external device (and therefore its user) communicating with the implant to include a MICS or MedRadio transceiver be within 2 meters of the implant may be difficult to achieve, or may result in suboptimal arrangement of the operatory suite to accommodate the 2 meter distance, or a non-MICS or non-MedRadio device may be desired to be utilized. This may be exacerbated for situations when interference or other conditions result in reducing the distance between the implant and the external device to maintain reliable communication. Due to equipment collocated in the operating room as well as other conditions affecting the strength of the connection between the implant and external device, a doctor or clinician may have to position themselves at a location less than 2 meters from the patient. This can encroach upon the working area required around the patient and possibly even the sterile field. Being able to effectively extend the communication range distance between the user interfacing with the implant beyond the 2 meter distance would in such situations be of great advantage.

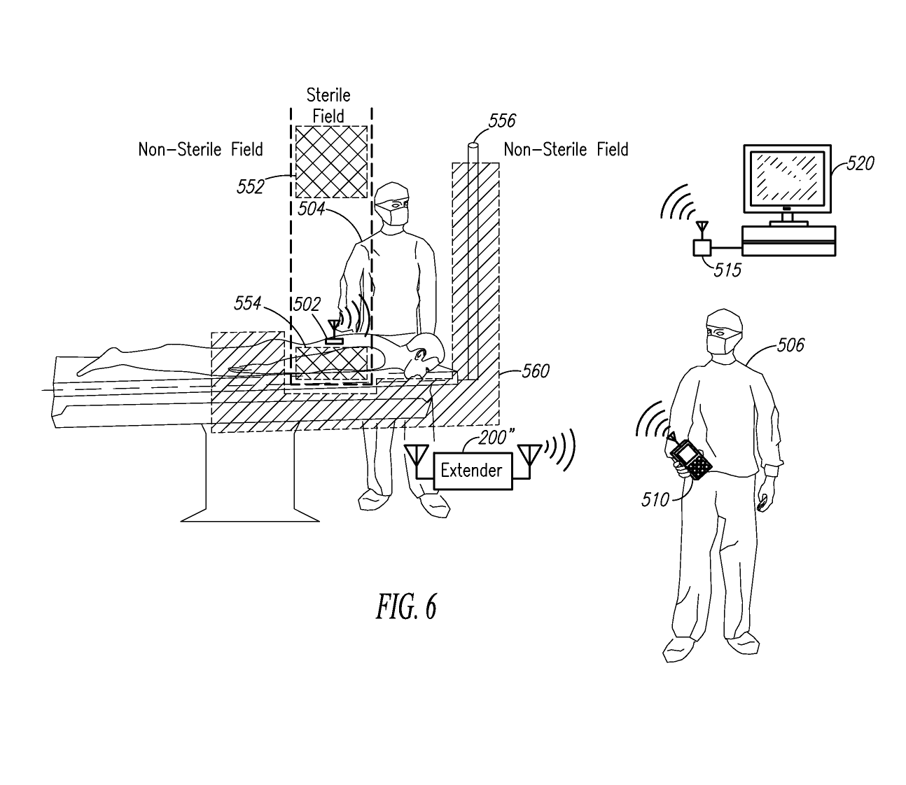

FIG. 6 shows an example of such a use, where an implanted medical device 502 is being implanted in a patent in a sterile operating room. The programmer 510 being used by a doctor or clinician in the operating room communicates with the implanted medical device 502. This programmer 510 might be MICS or MedRadio capable and if the clinician 506 can be positioned outside of the sterile field, but within 2 meters of the implanted medical device 502, and thus may be able to directly communicate with the implanted medical device 502. An extender 200″ can be used where the clinician operator 506 of the programmer 510 needs to be positioned in the non-Sterile field more than 2 meters from the implanted medical device 502 so as to not interfere with personnel 504 required within the sterile field. Alternatively or concurrently, the extender 200″ is useful if the programmer 510 uses f a non-MICS or non-MedRadio protocol, thus allowing communication with the implanted medical device 502. Furthermore, the extender 200″ can give a remote device 520, such as a PC, remote access to the implanted medical device even when the PC 520 is located outside of the sterile environment, or even outside of the operating room. In this case, the PC 520 communicates with the extender 200″ via a Wi Fi router or access point 515.

The programmer 520 will typically be operated by a clinician operator 506, but in some embodiments such programming could be automated by computer program, for example, eliminating the need for a human operator, in which case the PC 420 or some other computer or automated device, for example, may perform the programming function.

The extended range provided by the extender 200″ allows the doctor or clinician to position themselves in the operating room in potentially a less unobtrusive location. The extender 200″ can be physically located near or perhaps within the sterile field at locations 552 or 554, for example, but the doctor or clinician 506 is able to stay out of the way of essential operating room personnel and equipment. The extender 200″ is of much smaller and unobtrusive physical size and is more easily accommodated within this critical operating room area than is a person or some more extensive equipment.

The extender can then be used to facilitate external communication with the implanted medical device prior to or during surgery, and even directly after surgery while the patient in still in the operating room in order to check the operation and status of the medical device, and/or to program the device. For example, if the patient is conscious during surgery, various functions of the implanted medical device could be checked by testing various settings and monitoring the patient's response, or asking the patient for a response, during the test.

If the extender is to be located within the sterile field (e.g., at 552 or 554), then the extender itself can be sterilized, or the extender can be placed into a sterile enclosure, such as by using a sterile bag or pouch. Example sterilization methods for use with the extender include autoclaving, ethylene oxide (ETO) sterilization, chlorine dioxide (CD) gas sterilization, hydrogen peroxide sterilization, gamma ray sterilization, electron beam sterilization, and the like. Sterilization of the extender can take place locally at the hospital, or at a remote facility and delivered pre-sterilized to the hospital in sterilized packaging (similar to that which might be used for the implanted medical device). The extender can be returned to the remote facility, after being used during an operation, for example, for additional sterilization and reuse. Alternatively, the sterile bag or pouch can be utilized for sterilizing the extender for use in the sterile evironment. The operating room personnel would follow standard aseptic procedures to place the un-sterilized extender into the sterilized bag, seal it, and locate the extender within the sterile field. The sterile bag might be hung within the sterile field, such as from an IV pole 556 or bed rail within the sterile field, or attached directly to the operating table. The sterile bag should be chosen so that it has little to no affect on the communication capabilities of the extender (e.g., the bag should not block a wireless signal from the extender). After being used during an operation, the extender is removed from the bag for reuse in another operation, where another sterile bag would be used. The used bag can be disposed of as a biohazard.

Typically, the sterile field is an area outlined by placement of a sterile drape around the surgical site. The surgical drape establishes a horizontal area that may cover only a portion of the patient's body that starts at the level of the table/bed and extends vertically upwards. As an example, the procedure may only require that a sterile field be established covering an area from a patient's lower- to mid-back. The patient's hips and below, as well as shoulders and above, are located outside of the sterile field. Similarly, the area below the horizontal plane is defined as un-sterile. Therefore, the area on the side and beneath the table/bed may also be outside of the sterile field.

If the extender is to be located outside of the sterile field, such as at location 560, then once the sterile field has been defined, appropriate locations for the extender within close proximity to the implanted device can be identified. Example locations would be just outside of the sterile field (e.g., such as near the patient's hips or head). Another location is below the surface of the table/bed. However, care should be taken so that the table/bed itself does not interfere with the extender's communication capabilities. The extender can be quite small and compact and, therefore, can be readily placed so as to be free from interfering with other equipment, personnel or the patient.

The location of the extender can be fixed during an operation, so that it is not accidentally dropped or moved out of its communication range. For example, the extender might be taped or clamped to the table/bed, or attached using a hook-and-pile type fastener (e.g., Velcro).

Remote Care Applications

The ability to provide medical care at a distance (remote care) without the need to have a physical interaction between a caregiver, also referred to in this document as the clinician, and patient is an increasingly desired capability, particularly for medical device manufacturers. The ability to provide remote care offers many advantages to both caregiver and patient.

For example, it allows the patient to receive treatment without having to physically travel to the caregiver's location. While reducing cost and time required of the patient, it also has a significant impact for patients that have mobility problems whether as a result of age or physical condition, for which travel to the caregiver poses a considerable and arduous burden.

Receiving remote care can also provide a significant advantage to otherwise capable patients but who live at a considerable distance from the caregiver. Such a situation may not be all that uncommon, especially in instances where the care is somewhat specialized for which the number of providers may be limited and only found in major population centers.

For the caregiver, being able to provide remote care provides an increased level of flexibility in providing that care. Just like the patient, the caregiver is not required to meet with the patient at a specified location in a structured timeframe such as office hours. The care giver has the ability to provide care at a time and place of their choosing. This freedom can also reduce the cost of providing service in terms of maintaining a physical presence such as an office and support staff.

Remote care allows the caregiver to be accessible to a larger region of patients, increasing their patient base and revenue. It also allows the caregiver an alternative approach to addressing the needs of patients that can be addressed remotely rather than in the office, thereby leaving limited office schedules available for patients truly requiring a physical interaction. One aspect that could be opened by remote care is that in some applications, some number of otherwise unscheduled patients might be addressed outside of normal office hours, rather than having to try to fit in to be seen in an already full schedule, leading to quicker addressing of patient issues.

When the caregiver is a medical device manufacturer's field representative, the ability to remotely connect to a patient has many similar advantages to those already identified. For a medical device manufacturer, such a feature can allow fewer reps to cover a larger territory. A significant cost savings can be envisioned just for cutting travel.

The primary issue that could stand in the way of providing remote care compared to physical presence care is centered on the ability to address any concerns that require the care giver to take action if a patient comes under duress as a result of the remote administering of care, or specifically in this case, modifications to the operation of the patient's active medical device. This is the underlying reason why remote care up to this point is limited to primarily a monitoring (passive) model rather than a dispense (active) model. That is, operational information is obtained by transmission of data from the patient's device and made available to the care giver.

When dispensing, or making changes to the operation of a patient's device, it would be beneficial to have the ability to adequately address the risk that the patient may come under duress as a result in the change in operation of their device. Mitigating this risk can include providing a way to reduce or eliminate the cause of duress, if the patient was determined to be under duress, or that sufficient control of the patient's device was lost or otherwise lacking.

Discussed below is a method that addresses the need to mitigate the risk to a patient related to remote modification of their device's operation. In part of this method, an electronic device which bridges the different wireless communication technologies which might be used between the remote care giver and the patient is identified as well as functionality it may incorporate related to mitigating the risk of remote programming.

Initiation of Remote Programming

In order to allow remote programming to take place between the two parties outside of each other's physical presence, some means of audio, or audio/visual interaction should underlie the remote programming session between the clinician and patient. This may be accomplished through a variety of means, such as: landline phone, cell phone, internet, etc. Prior to moving forward with remote programming, the clinician needs to establish that the means used to verbally and possibly visually interact with the patient are sufficient to determine whether the patient is under duress as a result of programming.

When the clinician interfaces to a remote programming device he initially needs to establish a connection to the patient's implanted device through use of the extender.

Initial information may be requested by the clinician from the patient's implanted device. This initial information can include retrieval of the patient's implanted device identifying information (e.g., serial number, model number, MAC address, IP address, etc.) and current programming parameters.

Once the care giver verifies that the connected implanted device is the correct one to be modified, (the patient may have more than one implanted device, for example) the clinician may request additional information from the implanted device or issue commands to establish that the patient's implanted device is in proper working order possibly using diagnostic functions of the implanted device or logging reports.

After having determined that the patient's implanted device is in proper working order to allow for remote programming, modified implanted device programming is sent from the clinician's remote programming device through the extender to the patient's implanted device (this could occur using the Internet or a dedicated phone line, for example).

A response is sent back from the patient's implanted device through the extender and back to the remote programmer to indicate whether the programming was received and verified to be without error and is suitable for execution by the implanted device. A part of that determination may be that even though the implanted device received the modified programming information correctly, the operational parameters values may be checked against associated limit values for the parameter contained in the implanted device that were established prior for the patient, such as at an initial thorough programming session with the patient performed when caregiver and patient were in immediate interaction (physical presence) with each other.

After having verified the remote programming modifications are valid and executable, two example approaches to the primary concern with remote programming can be considered. Both include having an external device (e.g., the remote programmer used by the clinician) continuously send a continuously paced signal, hereafter referred to as a heartbeat, which must be received and processed by the implanted device in order for activity of the medical device such as stimulation, once started by the external device, to remain in effect. When operated for remote programming, the patient's implanted device monitors to ensure that the paced heartbeat signal is received within the expected timeframe. Timely reception allows stimulation to continue with remote programming values. Detecting that the heart beat signal has not been received within the expected timeframe will result in the patient's implanted device immediately disabling stimulation and getting to a known off state or reverting to a prior known operational mode.

A heartbeat time interval on the order of around to 15-30 seconds would be one practical example. This estimation is based on providing a repetitive input to the device (tapping a button) every 2-4 seconds, allotting some time to determine whether the patient is actually in duress (e.g., ask the patient “are you OK?”, wait for a response, determine no the patient in not OK), and then stop the heart-beat signal in the system, have it propagate through to the implant and transition to a safe mode of operation. This timeframe is likely similar to the situation where programming is done in the office.

Remote Heartbeat

In this approach, initiation and continued stimulation on the patient's implanted device is controlled by the clinician through the remote programmer while the connection is maintained.

Prior to sending a command from the remote programmer to the implanted device to initiate stimulation, the remote programmer may send a number of “pings”, or non-action messages through the extender to the implanted device. These messages may also include some method of establishing their sequence so that missing or dropped messages can be determined. These messages are used to establish the one-way time it takes to traverse the communication path from the remote programmer, through the extender, and to the implanted device as well as an indication of its quality.

The effect of these messages is to characterize the communication path transmission from the remote programmer, through the extender, to the implanted device. The information generated from this characterization allows the remote programmer to determine first, the quality of the communication path based on the number of dropped messages (if any), and second, the average and maximum time required to send a message from the remote programmer, through the extender, to the implanted device.

This information can be used to determine whether a “heartbeat” signal can be sent at an acceptable interval. Note that especially reliable networks (or network protocols) can be utilized, where available, to improve the chance of successful procedures.

When the clinician (or an evaluation program) has determined that the remote changes to the patient's implanted device and other conditions are suitable to allow stimulation to be initiated on the patient's implanted device, the clinician sends a command from the remote programmer, through the extender, to the patient's implanted device. The process of applying stimulation to the patient using the generator is initiated.

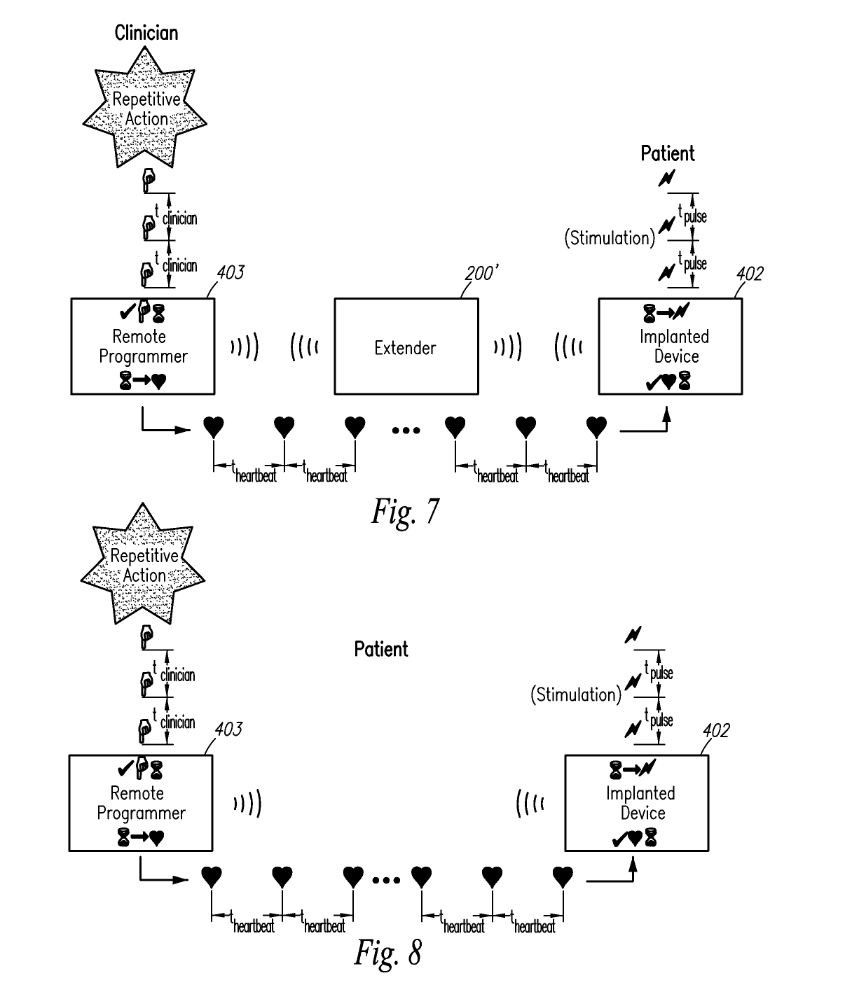

Immediately following (or prior to) the command to start stimulation, the clinician's remote programmer starts to send a heart beat message at a specified rate from the remote programmer 403, through the extender 200′ to the patient's implanted device 402. See FIG. 7.

While providing therapy, such as providing stimulation, the patient's implanted device requires that the heartbeat signal from the remote programmer be received within a set timeframe around the heartbeat rate. If the patient's implanted device does not receive the heartbeat signal within the allotted time frame, the patient's implanted device will immediately disable further stimulation and return to a safe state. Thus the purpose of the heartbeat is to establish a mechanism that ensures stimulation only continue when multiple critical conditions are all in a proper state or under control.

Since the heartbeat only allows stimulation to continue when received in the timeframe following the previous heartbeat, the heartbeat interval determines the amount of time that stimulation may continue without being in control. One factor to be considered in establishing a heartbeat interval is the amount of time considered acceptable for a patient to have their stimulation out of control. For example, the required time frame is chosen based on the therapy being provided by the medical device. Where problems in the therapy could cause severe or irreparable harm, small intervals are used, and the medical device quickly enters a safe state when the heartbeat is interrupted. However, in situations where the therapy is unlikely to cause any harm or serious discomfort, longer intervals could be chosen, and momentary interruptions in the heartbeat might be ignored.

Another practical understanding of this is its effect on the amount of time for which a patient remains under duress as a result of the remote programming before it can be removed. If this time is longer than the time required to send a message from the remote programmer, through the extender to the implanted device, then remote programming should probably not be allowed. If the message travel time is determined to be less than the allowed patient duress time, then a faster paced (lower) heartbeat interval can be used that will result in the patient being under duress for less time than otherwise allowed for the acceptable patient duress time period that would be allowed for the system.

The heartbeat interval to use for a given remote programming stimulation activation trial action can be specified by the remote programmer to the patient's implanted device before stimulation is activated.

This regularly issued heartbeat signal indicates that the connection between the remote programmer and the implant is intact. If the implant does not receive the continue signal in the time frame expected, it immediately disables further stimulation and returns to a safe state. Thus any substantial break in the communication connection between the remote programmer and the extender, or between the extender and the patient's implanted device can result, where desired, in the implanted device ceasing stimulation within the timeframe of the missed heartbeat.

Because the modifications to the patient's implanted device operating parameters being developed through remote programming have the potential to cause discomfort to the patient, or perhaps even carries some risk of harm, the remote programmer should have the ability to control stimulation and quickly stop it should the clinician detect concern for the patient. The clinician therefore preferably monitors the patient for duress whether through audio or audio/visual observation, such as by using a telephone connection, or audio or audio-visual teleconferencing capability. If at any time, the clinician determines, or is concerned that a patient is under duress, the clinician can cause the remote programmer to stop the sending of heartbeat messages.

In order to assure that the clinician himself is without duress, the clinician should continually provide distinct repetitive input to the remote programming device. This may mean continuous scrolling of a graphic input on the remote programmer user interface such as a wheel or other slide type control, or, it may be through requiring the user to repetitively tap a button on the user interface. This repetitive input is shown schematically in FIG. 7. The repetitive action is such that it does not impose on the clinician's ability to determine whether the patient is under duress.

To summarize, the heartbeat signal provides a unified means to help ensure that the following conditions are in place when evaluating remote programming modifications:
The connection between the remote programmer to the bridge;
The connection between the bridge to the generator;
A properly functioning remote programming device; and
An active clinician.

While the heartbeat mechanism described above outlines a process whereby the clinician modifies programming parameters, starts stimulation, assess change, stops stimulation, repeat, . . . , the heartbeat mechanism does not preclude programming modifications made in a more interactive manner, such that the changes are made while stimulation was active. A caveat to allowing such an operation is that the effects of transmitting and processing the programming parameter modifications should be considered for its affect on the heartbeat interval. For example, the implanted device design may be such that it can only process a single command or message at a time. If the time required to receive and process a change to programming takes longer than the allowed heartbeat interval, then the implanted device may determine that the heartbeat occurred outside of the allowed timeframe even though it had been received and queued into the implanted device's received message buffer.

Localized Heartbeat

Another approach to remotely programming the medical device would be to transfer the modified programming to the implanted device, then relinquish control of the implanted device to the patient rather than controlling it remotely. The patient then initiates activation of stimulation with the new programming parameters and monitors the stimulation effect as does the clinician (such as by audio or video, for example), except in this case the patient can affect continued stimulation but the clinician cannot. Or, in some instances, the clinician may be provided with an override function (or vice versa).

Similar in fashion to the heartbeat message sent by the remote programmer, the patient programmer 406 sends a regularly paced heartbeat message to the implanted device 402 in order for stimulation to continue. See FIG. 8. Also similar, a method to assure that the patient is not under duress as a result of the new implanted device programming is desirable as was described for the clinician above.

The patient programmer monitors for the distinct repetitive patient input in order to continue sending the heartbeat message. If the heartbeat message is not received by the patient's implanted device, either as a result of the patient not providing the distinct repetitive input, or some other issue as a result of the patient programmer operation, or due to the loss of the communication signal between the patient programmer and the implanted device, the implanted device immediately ceases stimulation.

In this case, the method used to determine whether the patient is under duress should consider the possible effects that stimulation may have on the patient's ability to operate the programming device. The patient can be required to provide a distinct repetitive input to the patient programming device in order to establish that the patient is not under duress. This is contrasted to another method whereby the patient might be required to press and hold a button in order to maintain stimulation. It is possible, especially considering that stimulation might affect muscle control, that the patient might continue to push a button as a result of the stimulation forcing contraction of muscles such that they cannot release the button when under duress. Requiring repetitive input from the patient establishes that they are in not likely in duress and have control of their programming device.

Modal Heartbeat

As described above, a heartbeat message is regularly received in order for stimulation to remain active. For use of the patient programmer, the majority of use is such that the patient establishes communication with the implanted device, initiates stimulation with the intent that it continue to be applied even after the communication path between the programming device and the implanted device is terminated. This is done for reasons of saving power on both the patient programming device and implanted device as a continuous active communication connection between the two is not required.

The programming of the implanted device device is such that requiring the heartbeat message is conditional upon the intended functional operation of the implanted device for normal or remote programming. Commands and operational modes therefore should, in most instances, take this into account.

The extender can be utilized for supporting various other functions related to the implanted medical device. For example, the physical location of the end user with the implanted medical device might be some distance away from a desired connection—for example, a patient with a MICS or MedRadio implant may live in a remote location and a clinician desires to access the patient's implant without the patient making a trip to the clinician's office. Or the end user connecting to the implant might not even be a person, but a software application used to monitor the implant or collect implant operation data. In such situations, it may be desirable to use commercial-off-the-shelf (COTS) components. However, in such situations, requiring the external device or COTS to incorporate a MICS or MedRadio transceiver incurs a significant cost for the development of hardware and software to include a MICS or MedRadio transceiver.

Instead, the extender could be utilized to connect the implanted medical device to the remote device via the Internet, such as in a manner discussed above (e.g., connecting to the Internet via a router). This can allow a general purpose PC to be used as the external device. As discussed above, the extender allows a user or other entity to use an end device that does not incorporate a MICS or MedRadio transceiver as a means to control or otherwise exchange data with a medical implant that incorporates MICS or MedRadio.

A common 802.11 wireless home network (i.e., Wi Fi) or an Ethernet connection could be used to allow mobility for a patient at home with communication to another device on the local network or on an external network. Smartphones or other portable devices incorporating communication capabilities such as Bluetooth, Wi Fi, etc. could be used as a means to provide remote (long distance) monitoring of implant operation.

The extender can also provide a valuable platform from which to base device testing of the implant (software, EMC, etc.) or clinical research for use of existing implant devices (animal testing, etc.) prior to implantation in humans.

To provide benefits to the implant patient, a Smartphone application could be used to allows a patient to interface with their implant using a device they are already familiar with, and are likely to have on their person, such as a cell phone, PDA, or pad device, for example, avoiding the need to make such devices MICS or MedRadio compatible.

Many other example embodiments of the invention can be provided through various combinations of the above described features. Although the invention has been described hereinabove using specific examples and embodiments, it will be understood by those skilled in the art that various alternatives may be used and equivalents may be substituted for elements and/or steps described herein, without necessarily deviating from the intended scope of the invention. Modifications may be necessary to adapt the invention to a particular situation or to particular needs without departing from the intended scope of the invention. It is intended that the invention not be limited to the particular implementations and embodiments described herein, but that the claims be given their broadest reasonable interpretation to cover all novel and non-obvious embodiments, literal or equivalent, disclosed or not, covered thereby.

What is claimed is:

1. An apparatus for providing communication between an implant in a sterile environment and an external device, comprising:
a first transceiver for wireless communication with said implant using a first transmission protocol; and
a second transceiver for wireless communication with said external device using a second transmission protocol, wherein
said apparatus is adapted for connecting said implant to said external device by bridging said first protocol and said second protocol, and wherein
said first transmission protocol is a protocol restricted to a short range near the implant, and further wherein
said apparatus is adapted to be sterilized for placing in the sterile environment.

2. The apparatus of claim 1, wherein said first protocol is a MedRadio protocol.

3. The apparatus of claim 2, wherein said second protocol is a Bluetooth or Wi Fi protocol.

4. The apparatus of claim 2, wherein said short range is a range that is about 2 meters or less.

5. The apparatus of claim 1, further comprising a sterilized bag, such that said bag is adapted to sterilize said apparus for use in the sterile environment.

6. The apparatus of claim 1, wherein said apparatus is adapted to be sterilized by exposing said apparatus to a sterilizing environment without harm to said apparatus.

7. An apparatus for providing communication between an implant using a short-range medical device communication protocol and a remotely located external device, comprising:
a first transceiver for wireless communication with said implant using a first transmission protocol; and
a second transceiver for communication with said external device using a second transmission protocol over a communication network, wherein
said apparatus is adapted for connecting said implant to said external device by bridging said first protocol and said second protocol, and wherein
said first transmission protocol is a protocol restricted to a short range near the implant, and further wherein
said apparatus is adapted to be sterilized for placing in the sterile environment.

8. The apparatus of claim 7, further comprising a sterilized bag, such that said bag is adapted to sterilize said apparatus for use in the sterile environment.

9. The apparatus of claim 7, wherein said apparatus is adapted to be sterilized by exposing said apparatus to a sterilizing environment without harm to said apparatus.

10. An apparatus for providing communication between an implant using a short-range medical device communication protocol and a remotely located external device, comprising:
a first transceiver for wireless communication with said implant using a first transmission protocol; and
a second transceiver for communication with said external device using a second transmission protocol over a communication network, wherein
said apparatus is adapted for connecting said implant to said external device by bridging said first protocol and said second protocol, and wherein
said first transmission protocol is a protocol restricted to a short range near the implant, and further wherein
said apparatus is adapted to communicate a heartbeat signal between said implant and said external device.

11. A system for updating a program in a medical device, comprising:
a medical device for providing therapy to a patient;
a programming device adapted for remotely programming a medical device; and
an apparatus for providing communication between said medical device using a short-range medical device communication protocol and said programming device located remotely from said medical device, comprising:
a first transceiver for wireless communication with said medical device using a first transmission protocol, and
a second transceiver for communication with said programming device using a second transmission protocol over a communication network, wherein
said apparatus is adapted for connecting said medical device to said programming device by bridging said first protocol and said second protocol, and wherein
said first transmission protocol is a protocol restricted to a short range near the medical device;
wherein, said system is adapted to provide a heartbeat signal between said medical device and said remotely located programming device; and wherein
said medical device is adapted to enter a safe mode during remote programming by said remotely located programming device if said heartbeat signal is corrupted, lost, or otherwise interrupted for a defined period.

12. The system of claim 11, wherein said medical device is implanted in the body of a patient for providing said therapy.

13. The system of claim 12, wherein said therapy provides stimulation pulses to one or more internal structures of the patient.

14. The system of claim 11, wherein said communication network is the Internet.

15. A method for bridging communication between an implant using a medical device communication protocol in a sterile environment and an external device using a second communication protocol outside of said sterile environment, comprising the steps of:
sterilizing a bridging device comprising a first transceiver for wireless communication with said implant using said medical device communication protocol and also comprising a second transceiver for wireless communication with said external device using a second transmission protocol;
placing the sterilized bridging device in or near the sterile environment near the implant; and
using the bridging device for providing a wireless communication path between said implant and said external device that is outside of said sterile environment.

16. A method for bridging communication between an implanted medical device and a communication device, comprising the steps of:
placing a bridging device near a patient having a surgical procedure, wherein said patient has an implanted medical device embedded or being embedded in the patient;
the bridging device wirelessly communicating with the implanted medical device during or just after the surgical procedure; and
the bridging device also wirelessly communicating with a communication device provided outside of said sterile environment, wherein
the bridging device wireless communication provides a wireless communication path between said implant and said communication device that is outside of said sterile environment.

17. The method of claim 16, further comprising the step of sterilizing the bridging device prior to placing said bridging device near the patient.

18. The method of claim 17, wherein said sterilizing step includes the step of placing the bridging device in a sterile package.

19. The method of claim 17, wherein said sterilizing step includes the step of treating the outer surface of the bridging device to sterilize said outer surface.

20. A method of remotely programming a medical device, comprising the steps of:
establishing a communication path between the medical device and the remotely located programming device;
establishing a heartbeat between said medical device and said remotely located programming device;
remotely programming said medical device; and
monitoring said heartbeat at said medical device such that if said heartbeat is corrupted, lost, or otherwise interrupted for a defined period, said medical device enters a safe mode.

21. The method of claim 20, wherein said medical device is implanted in the body of a patient.

22. The method of claim 20, wherein said medical device is used to provide stimulation pulse therapy to a patient.

23. The method of claim 20, wherein said step of remotely programming said medical device includes the step of updating parameters in said medical device for changing a therapy provided to a patient by said medical device.

24. A method of remotely programming a medical device, comprising the steps of:
establishing a communication path between the medical device and the remotely located programming device;
establishing a heartbeat between said medical device and said remotely located programming device;
remotely programming said medical device;
establishing a communication link between a patient receiving therapy from said medical device and a clinician operating said programming device such that the clinician can monitor a condition of the patient while programming said medical device;
monitoring said heartbeat at said medical device such that if said heartbeat is corrupted, lost, or otherwise interrupted for a defined period, said medical device enters a safe mode; and
providing a mechanism for the clinician to cause said medical device to enter the safe mode by manual activation by the clinician.

25. A method of providing therapy to a patient, comprising the steps of:
providing an ambulatory medical device to the patient for use by the patient for providing therapy to the patient;
establishing a communication path between the medical device being used by the patient and a remotely located programming device that is not operated by the patient; and
remotely programming said medical device for updating said therapy.

26. A method of providing therapy to a patient, comprising the steps of:
providing an ambulatory medical device to the patient for use by the patient for providing therapy to the patient;
establishing a communication path between the medical device being used by the patient and a remotely located programming device that is not operated by the patient;
remotely programming said medical device for updating said therapy
establishing a heartbeat between said medical device and said remotely located programming device; and
monitoring said heartbeat at said medical device such that if said heartbeat is corrupted, lost, or otherwise interrupted for a defined period, said medical device enters a safe mode.

27. The method of claim 26, further comprising the steps of:
establishing a communication link between a patient receiving therapy from said medical device and the remote location for monitoring a condition of the patient while programming said medical device; and
providing a mechanism for causing said medical device to enter the safe mode by activation from the remote location.

28. A method of providing therapy to a patient, comprising the steps of:
implanting a medical implant in a patient where at least a portion of the patient is in a sterilized environment;
providing a bridging device in or near the sterilized environment, said bridging device comprising a first transceiver for wireless communication with said implant using a first communication protocol and also comprising a second transceiver for wireless communication with an external device using a second communication protocol;
placing said external device outside of said sterile environment; and
using the external device for programming a therapy into said medical implant while said at least a portion of the patient is in said sterile environment by using said bridging device for providing a wireless communication path between said implant and said external device that is outside of said sterile environment.
